(19) United States
(12) Patent Application Publication
(10) Pub. No.: US 2026/0096748 A1
(43) Pub. Date: Apr. 9, 2026

(54) ANOMALY DETECTION FOR COMPRESSION

(71) Applicants: Dexcom, Inc., San Diego, CA (US); UNIVERSITÀ DEGLI STUDI DI PADOVA, Padova (IT)

(72) Inventors: Andrea Facchinetti, Padova (IT); Simone Del Favero, Padova (IT); Giovanni Sparacino, Padova (IT); Elena Idi, Padova (IT); Eleonora Manzoni, Padova (IT)

(21) Appl. No.: 18/907,825

(22) Filed: Oct. 7, 2024

(51) Int. Cl. A61B 5/145; A61B 5/00

(52) U.S. Cl. CPC A61B 5/14532; A61B 5/7264

(57) ABSTRACT

An unsupervised method of detecting a pressure induced sensor artifact (PISA) in an analyte signal includes receiving an analyte trace having a plurality of data samples obtained over a period of time. A plurality of predetermined features reflective of a PISA in the analyte trace is extracted to produce a plurality of data points in a feature space defined by the plurality of predetermined features. The data points in the feature space are analyzed using an unsupervised anomaly detection algorithm to produce an anomaly score for each of the data points. Data points that have an anomaly score that exceeds a threshold are identified. An alert is generated indicating that a PISA is present in data samples associated with the data points that have an anomaly score that exceeds the threshold.

### BACKGROUND

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also potential. Diet and exercise also affect blood glucose levels.

Diabetes conditions are sometimes referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become insulin resistant. The result is that even though insulin is present in the body, the insulin is not sufficiently used by the patient's body to effectively regulate blood sugar levels.

Management of diabetes can present complex challenges for patients, clinicians, and caregivers, as a confluence of many factors can impact a patient's glucose level and glucose trends. To assist patients with better managing this condition, portable or wearable medical devices (e.g., sensors and other types of monitoring and diagnostic devices) as well as a variety of diabetes intervention software applications (hereinafter “applications”) have been developed by various providers.

Because continuous glucose monitoring (CGM) sensors are able to measure glucose concentration in a user, they are important for diabetes management. Analysis of CGM data is beneficial for improving diabetes therapies and, in turn, for improving overall glycemic control for a user. For example, analysis of trends and patterns in CGM data after a meal can help improve insulin dosing by suggesting rescue carbohydrate intake, evaluating glucose variability, quantifying the effectiveness of a therapy, and improving visualization of relevant CGM patterns.

Unfortunately, CGM traces can be inaccurate. One source of uncertainty arises from random measurement error which affects any signals obtained from a sensor. Another source of inaccuracy is that caused by large artifacts that can be occasionally generated during sensor functioning. FIG. 1 (left) shows a simulated example of a PISA in CGM data and FIG. 1 (right) shows an example of a CGM trace corrupted by a fault that occurs in the shaped portion of the CGM trace. displays a portion of representative CGM data clearly affected by both these sources of error.

The presence of large artifacts in CGM traces can induce errors of clinical relevance. For instance, the spurious drop in BG highlighted in FIG. 1 (right) by the shaded area can be misinterpreted as a hypoglycemic event. Therefore, the preliminary detection and elimination of CGM data portions affected by errors and artifacts is important to minimize the risk of making incorrect clinical evaluations and therapy decisions.

The glucose measurement in CGM sensors is typically performed by a glucose-oxidase reaction on a small needle, usually inserted in the subcutaneous tissue of the abdomen or the upper back of the arm. Failures on the CGM sensor measurements are often related to the biomechanics of the sensor-tissue interface. Specifically, the mechanical compression of the sensor, for example triggered by the individual who unintentionally applies pressure on the sensor during sleep, alters the diffusion process at the needle insertion site. This alteration adversely affects the sensor's sensitivity, leading to a systematic underestimation of glucose concentration across multiple samples. The artifacts resulting from mechanical compression are commonly referred to as Pressure Induced Sensor Artifacts (PISAs). The drop in glucose levels that typically occurs at the beginning of PISA can be easily mistaken for a physiological fluctuation, making PISAs' detection a complex task, even for an expert human operator.

Studies have reported that pressure over the sensor insertion site led to a transient decrease in current. Notably, compression at the sensor insertion site has been implicated as a potential cause for anomalous hypoglycemic measurements, particularly during nighttime when users lie directly on the sensor. Moreover, many studies concerning animal trials illustrate acute pressure effects on CGM data.

Strategies aimed at addressing PISAs detection can be broadly categorized into two groups: data driven and model-based techniques. Model-based fault detection techniques use explicit models of patient physiology to predict the expected BG level under normal conditions. Data-driven approaches mainly exploit the availability of historical data and can be used for a variety of monitored processes. The performance of data-driven approaches heavily relies on training data, and possibly on labeled examples of faulty and fault-free data previously evaluated by an expert.

Data-driven approaches can be divided into supervised and unsupervised approaches. Supervised approaches require labeled data to train the algorithm. This is a significant limitation for its use in PISA detection, where labels must be assigned by an expert human operator based on visual inspection of the trace, a complex and time-consuming procedure, prone to errors and uncertainties. Labelled data are therefore scarce and often rather inaccurate. On the other hand, unsupervised approaches do not require labeled data and learn from unlabeled data.

### BRIEF DESCRIPTION OF THE DRAWINGS

FIG. 1 (left) shows a simulated example of a PISA in CGM data and FIG. 1 (right) shows an example of a CGM trace corrupted by a fault that occurs in the shaped portion of the CGM trace.

FIG. 2 is a diagram conceptually illustrating an example continuous analyte monitoring system including example continuous analyte sensor(s) with sensor electronics, in accordance with certain aspects of the present disclosure.

FIG. 3 is a flowchart showing one example of a method for detecting PISAs in measured analyte trace, in accordance with certain aspects of the present disclosure.

FIG. 4 illustrates five example features that may be extracted from a CGM signal to detect PISAs.

FIG. 5 shows illustrative false positive (FP)/day-recall curves using the training dataset.

FIG. 6 shows an example of a normalized representation of portions of a CGM trace detected with the Isolation Forest algorithm using a training dataset of simulated data.

FIG. 7 shows a boxplot of the population distributions of the recall and FP/day metrics in a test dataset.

FIG. 8 shows boxplots of the distribution of the recall and FP/day metrics in patients.

### DETAILED DESCRIPTION

Embodiments described herein detect Pressure Induced Sensor Artifacts (PISAs) using unsupervised algorithms since they do not require labeled datasets. To detect the PISAs, features of the artifacts in a CGM trace that distinguish them from unaffected portions of the CGM trace are extracted. Although the PISA detection techniques presented herein are described for illustrative purposes in terms of the detection of PISAs in CGM data obtained from a CGM sensor, more generally the systems and techniques described herein are applicable to the detection of PISAs in other analyte data obtained from other types of analyte sensors.

Accordingly, the embodiments described herein provide systems and methods of detecting PISAs in analyte data. In particular, the embodiments herein provide a health management system, including a display device and an analyte monitoring system, including an analyte sensor (e.g., CGM sensor) configured to generate analyte measurements (e.g., glucose measurements) for transmission to the display device. The display device includes a processor configured to execute a software application for receiving and processing the analyte data (e.g., CGM data) indicative of the analyte measurements generated by the analyte sensor. The software application may use a PISA detection algorithm to detect PISAs in received analyte data. The software application may alternatively be configured to send the received analyte data to a server that executes the PISA detection algorithm.

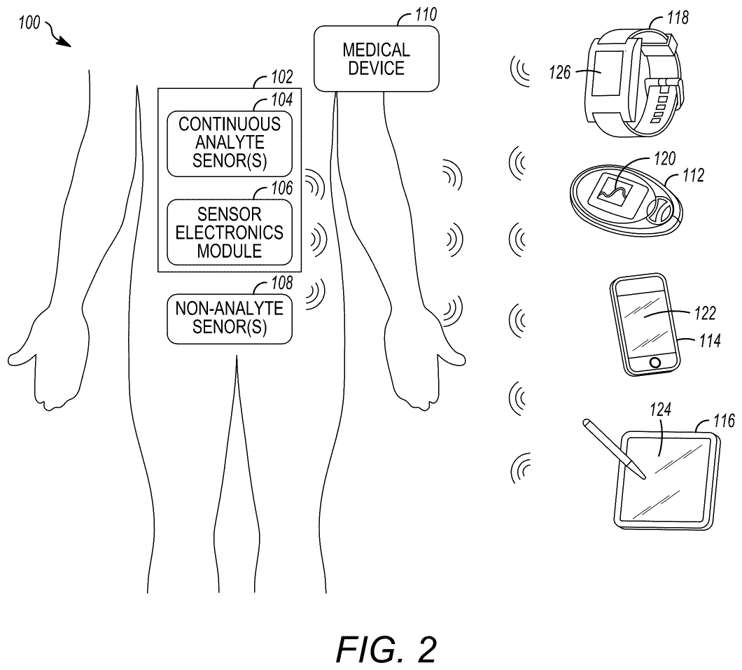

FIG. 2 illustrates an analyte monitoring system 100 including an example continuous analyte sensor system 102, non-analyte sensor(s) 108, medical device 110, and a plurality of display devices 112, 114, 116, and 118, in accordance with certain aspects of the present disclosure. The components of the analyte monitoring system 100 are configured to operate continuously to monitor one or more analytes of a user, in accordance with certain aspects of the present disclosure.

Continuous analyte monitoring system 102, in the illustrated embodiment, includes sensor electronics module 106 and one or more continuous analyte sensor(s) 104 (individually referred to herein as continuous analyte sensor 104 and collectively referred to herein as continuous analyte sensors 104) associated with sensor electronics module 106. Sensor electronics module 106 may be in wireless communication (e.g., directly or indirectly) with one or more of display devices 112, 114, 116, and 118. In certain embodiments, sensor electronics module 106 may also be in wireless communication (e.g., directly or indirectly) with one or more medical devices, such as medical devices 110 (individually referred to herein as medical device 110 and collectively referred to herein as medical devices 110), and/or one or more other non-analyte sensors 108 (individually referred to herein as non-analyte sensor 108 and collectively referred to herein as non-analyte sensor 108).

In certain embodiments, a continuous analyte sensor 104 may comprise a sensor for detecting and/or measuring analyte(s). The continuous analyte sensor 104 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 104 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 104 provides a data stream indicative of the concentration of one or more analytes in the user. The data stream may include raw data signals, which may then be converted into a calibrated and/or filtered data stream used to provide estimated analyte value(s) to the user.

In certain embodiments, continuous analyte sensor 104 may be a multi-analyte sensor, configured to continuously measure multiple analytes in a user's body. For example, in certain embodiments, the continuous multi-analyte sensor 104 may be a single multi-analyte sensor configured to measure two or more of glucose, insulin, lactate, ketones, pyruvate, and potassium in the user's body.

In certain embodiments, the continuous analyte sensor 104 may be a continuous glucose monitor (CGM). Some examples of a continuous glucose monitor include a glucose monitoring sensor. In some embodiments, glucose monitoring sensor is an implantable sensor, such as described with reference to U.S. Pat. No. 6,001,067 and U.S. Patent Publication No. US-2011-0027127-A1. In some embodiments, the glucose monitoring sensor is a transcutaneous sensor, such as described with reference to U.S. Patent Publication No. US-2006-0020187-A1. In yet other embodiments, the glucose monitoring sensor is a dual electrode analyte sensor, such as described with reference to U.S. Patent Publication No. US-2009-0137887-A1. In still other embodiments, the glucose monitoring sensor is configured to be implanted in a host vessel or extracorporeally, such as the sensor described in U.S. Patent Publication No. US-2007-0027385-A1. These patents and publications are incorporated herein by reference in their entirety.

As used herein, the term “continuous” may mean fully continuous, semi-continuous, periodic, etc. Such continuous monitoring of analytes is advantageous in diagnosing and staging a disease given the continuous measurements provide continuously up to date measurements as well as information on the trend and rate of analyte change over a continuous period. Such information may be used to make more informed decisions in the assessment of glucose homeostasis and treatment of diabetes.

In certain embodiments, sensor electronics module 106 includes electronic circuitry associated with measuring and processing the continuous analyte data, including prospective algorithms associated with processing and calibration of the analyte data. Sensor electronics module 106 can be physically connected to continuous analyte sensor(s) 104 and can be integral with (non-releasably attached to) or releasably attachable to continuous analyte sensor(s) 104. Sensor electronics module 106 may include hardware, firmware, and/or software that enables measurement of levels of analyte(s) via a continuous analyte sensor(s) 104. For example, sensor electronics module 106 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 112, 114, 116, and/or 118 are configured for displaying displayable analyte data, including analyte data, which may be transmitted by sensor electronics module 106. Each of display devices 112, 114, 116, or 118 can include a display such as a touchscreen display 120, 122, 124, or 126 for displaying analyte data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating analyte data to the user of the display device and/or receiving user inputs.

As described above, an analyte monitoring system 102 such as a CGM system is configured to continuously measure one or more analytes (e.g., glucose in a CGM system) and transmit the resulting analyte measurements, in the form of analyte data, to a display device (e.g., display device 112, 114, 116, and/or 118), which is configured with a PISA detection algorithm to detect PISAs associated with the analyte data before the analyte data is displayed to the user and/or analyzed for generating decision support recommendations. In certain embodiments, instead of a display device, the PISA detection algorithm may be executed on a server in data communication with the display device and/or continuous analyte monitoring system. In such embodiments, the server uses the PISA detection algorithm and transmits the results to the display device. In some embodiments, algorithms described herein may be implemented wholly or in-part on the display device, a server, and/or another device in communication with the display device and/or server.

### Fault Detection Method

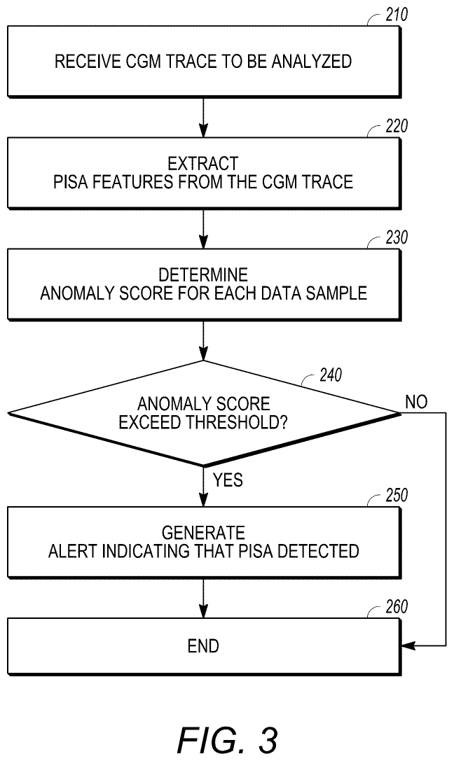

FIG. 3 is a flowchart showing one example of a method for detecting PISAs in measured analyte trace, which for illustrative purposes will be described in more detail below for the particular case where the analyte is glucose. At block 210 a CMG trace that is to be analyzed is received. Next, at block 220, the extraction of suitable features is obtained from the CGM trace to highlight the differences between normal instances of the data and the failures of the sensor. Illustrative features that may be extracted will be discussed in more detail below. At block 230 an anomaly detection (AD) algorithm, based on the features defined at the previous block, computes for each data sample in the trace an Anomaly Score (AS), which measures by how much the data sample differs from other data samples that are observed. The specific criteria employed to assign the score varies from one anomaly detection algorithm to another and will be discussed in more detail below. Finally, anomaly scores that are indicative of PISAs cause an alert such as an alarm to be generated. This may be accomplished at block 240, for example, by comparing the anomaly score to a previously determined threshold and generating an alert at block 260 if the anomaly score is determined to exceed the threshold at block 250. Examples of techniques for determining the optimal threshold will be discussed below.

### Feature Extraction

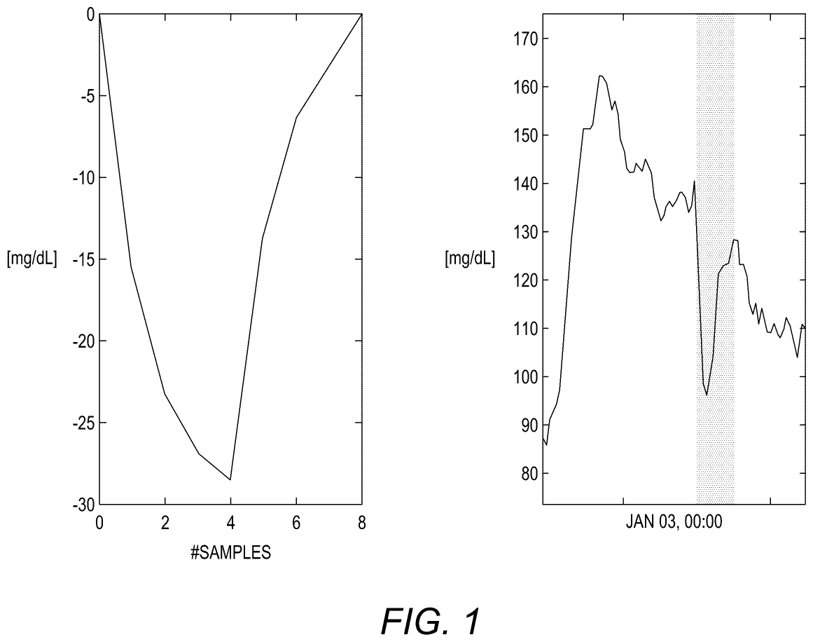

The typical form of a PISA is shown in FIG. 1. Due to the pressure applied to the sensor, the failures are characterized by an initial quick drop in blood glucose, followed by a rapid rebound to a normal reading once the episode concludes. To facilitate the episode recognition, the features are designed to emphasize these patterns in the CGM trace.

Because of the underestimation of the glucose level occurring during a compression artifact, failures are often associated with local minima and the value of a faulty data sample is expected to be lower than the neighboring samples. To highlight this characteristic the deviation of a glucose measurement from the average of its neighbor samples is calculated. More precisely, at each time step t, the feature:

$${f}_{1} ( t ) = \overline{CGM} ( t ) - CGM ( t ) \qquad ( 1 )$$

is computed, with

$$CGM ( t ) = \frac{1}{2 w + 1} {\sum }_{i = t - w}^{t + w} CGM ( i )$$

where w is the number of previous and subsequent samples considered. In other words CGM(t) is the average on a moving window with center in t and duration L=2w+1 samples. This feature will be referred to herein as the Deviation from the Local Values (DLV) feature.

PISAs are also characterized by the presence of a rapid decrease in the glucose concentration, followed by a fast recovery. This suggest considering a feature that is based on the derivatives of the CGM(t) signal. For instance, the first derivative of the CGM signal is estimated as

$${d}^{′} CGM ( t ) = \frac{1}{{T}_{s}} [ CGM ( t + 1 ) - CGM ( t ) ] .$$

where Tₛ=5 min is the sampling time. Then, the average absolute value of the derivatives computed in a moving window (centered in t with duration 2w+1) is calculated as:

$$\frac{1}{2 w + 1} {\sum }_{i = t - w}^{t + w} \vert {d}^{′} CGM ( t ) \vert .$$

This value is expected to be large when the signal is rapidly changing, such as during a fault, but it may also exhibit a persistent decrease or a sustained rise (for example, after a meal) in non-faulty portions of the CGM signal. To differentiate between these two situations, the second feature f₂(t) is computed as:

$${f}_{2} ( t ) = \frac{1}{2 w + 1} [ {\sum }_{i = t - w}^{t + w} \vert {d}^{′} CGM ( i ) \vert - \vert {\sum }_{i = t - w}^{t + w} {d}^{′} CGM ( i ) \vert ]$$

where the second term reduces the feature value in case of persistent increasing or decreasing data. This feature will be referred to herein as the Large Non-Monotonic Variations (LNMV) feature.

Next, a third feature may be introduced to highlight samples with strongly negative (or positive) dcgm(t) when compared with their neighbors:

$${f}_{3} = {d}^{′} cgm ( t ) - \frac{1}{2 w + 1} \vert {\sum }_{i = t - w}^{t + w} {d}^{′} CGM ( i ) \vert \qquad ( 2 )$$

This feature will be referred to herein as the Deviation from the Local Trends (DLT) feature.

A fourth feature may be introduced to detect portions of the CGM trace undergoing a rapid decrease followed by a rapid increase, as exhibited by PISAs. At first the positive and negative components are decoupled by considering the quantities:

$${\vert {d}^{′} CGM ( t ) \vert}_{> 0} = \{ \begin{matrix}{d}^{′} CGM ( t ) & if dCGM ( t ) \geq 0 \\ 0 & if dCGM ( t ) < 0\end{matrix}$$

$${\vert {d}^{′} CGM ( t ) \vert}_{< 0} = \{ \begin{matrix}0 & if dCGM ( t ) \geq 0 if \\ {d}^{′} CGM ( t ) & if dCGM ( t ) < 0\end{matrix}$$

Then, the average value of these two signals in two different moving window of length Z and separated by d samples is considered:

$${s}_{1} ( t ) = \frac{1}{L + 1} {\sum }_{i = t}^{t + L} {\vert {d}^{′} CGM ( t ) \vert}_{< 0}$$

$${s}_{2} ( t ) = \frac{1}{L + 1} {\sum }_{i = t + d}^{t + d + L} {\vert {d}^{′} CGM ( t ) \vert}_{> 0} .$$

Finally, the feature f₄(t) is implemented as the product of the two signals, s₁(t) and s₂(t):

$${f}_{4} ( t ) = {s}_{1} ( t ) {s}_{2} ( t ) .$$

This feature is large if and only if both s₁(t) and s₂(t) are simultaneously large at time t, and this happens only if there is a large decrease in the CGM signal followed by a large increase after d data samples. The parameters L and d are fixed in accordance with the duration of the failures: L=d=3. This feature will be referred to herein as the First-Down-Then-Up (FDTU) feature.

Finally, another feature f₅(t) is based on the second derivative of the CGM trace d″CGM(t). In particular, its average absolute value in a moving window centered in tis considered:

$${f}_{5} ( t ) = \frac{1}{2 w + 1} {\sum }_{i = t - w}^{t + w} \vert {d}^{″} CGM ( t ) \vert . \qquad ( 3 )$$

This feature is large in the presence of highly irregular data portions with frequent variations of the signal derivative. This feature will be referred to herein as the Concavity (Conc) feature.

As a final step all the unnormalized feature are normalized with minimum-maximum normalization.

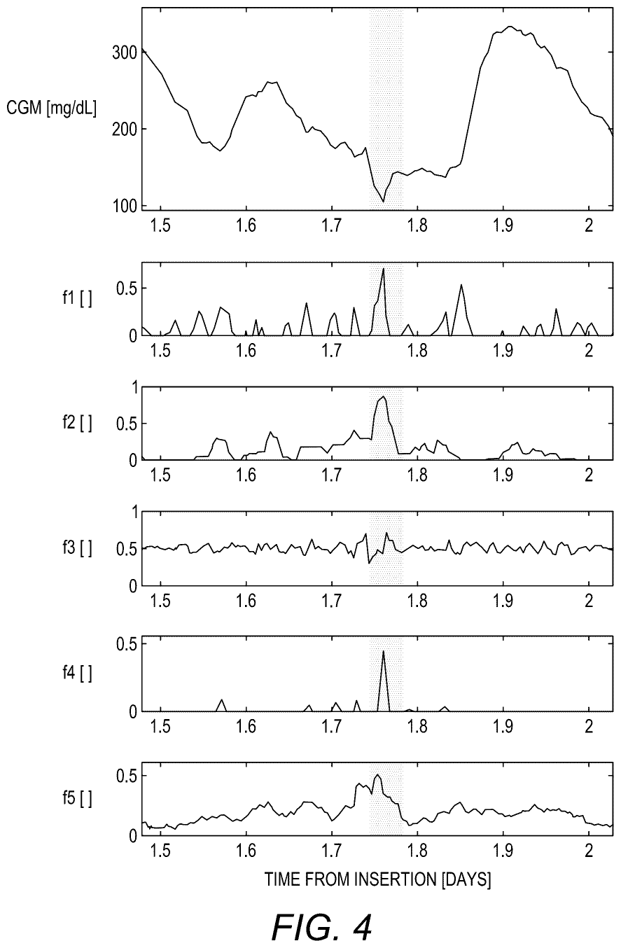

FIG. 4 illustrates the feature extraction process: starting from with a CGM trace, the five aforementioned features are extracted and normalized with minimum-maximum normalization. As the figure shows, the features in proximity of the fault (gray band), assume a higher value with respect to data points not impacted by a fault.

It should be noted that the five features discussed above are illustrative only and that alternative features may be used instead of or in addition to the features discussed above. Moreover, different embodiments may employ a different number of features in different combinations. For instance, in some embodiments one, two, three or more features may be employed for feature extraction to detect PISAs.

### Anomaly Detection Algorithms

To detect pressure-induced faults, unsupervised anomaly detection algorithms are employed. These techniques have been employed for a variety of different purposes, such as network security or forensics, by the machine learning community with the aim of finding rare items, events or observations which differ significantly from the general distribution of a population. By performing a multi-variate analysis in feature space, these methods produce an anomaly score for each data sample: the higher the score, the higher the probability that the data point is an anomaly.

The unsupervised anomaly detection algorithms can be classified into four main groups: (1) Nearest-neighbor based techniques, (2) Clustering-based methods, (3) Statistical algorithms and (4) Random Partitioning-based strategies. Table I lists algorithms that were used to illustrate the PISA detection techniques described herein. These algorithms we implemented using PyOD, which is an open-source Python toolbox. As an illustrative example of the functioning of these algorithms, the two algorithms that proved most effective (Isolation Forest and Histogram-Based Outlier Score) are briefly reviewed below. Of course, more generally, the PISA detection techniques described herein may employ a wide variety of other unsupervised anomaly detection algorithms as well.

TABLE I
COMPARISON OF RESULTS ACHIEVED
ON THE SIMULATED TEST SET

| Algorithm | Recall [ ] | FP/day [ ] |
|---|---|---|
| Isolation Forest | 0.74 | 0.17 |
| Histogram-Based | 0.70 | 0.23 |
| Outlier Score | | |
| Local Outlier Factor | 0.70 | 0.19 |
| [59] | | |
| Connectivity Outlier | 0.70 | 0.19 |
| Factor | | |
| Principal Component | 0.63 | 0.17 |
| Analysis | | |
| One-Classe Support | 0.61 | 0.20 |
| Vector Machine | | |
| K-Nearest Neighbors | 0.60 | 0.27 |

An illustrative unsupervised anomaly detection algorithm that was employed is the Isolation Forest algorithm, which looks for anomalies by leveraging space partitioning: an isolated point (anomaly) requires on average less iterations than an inlier to be isolated. Therefore, a simplified version of the algorithm works as follows: given a dataset X⊂R^{F}, where F is the number of features, an ensemble of T particular binary trees, called iTrees, is considered.

Each tree is built according to the following steps:
1) a sub-sample of data with cardinality ψ is randomly selected
2) a feature i∈[1, . . . , F] is randomly selected
3) a random threshold

$${x}_{i}^{*}$$

is set in the range

$$\{ {x}_{i}^{min} , {x}_{i}^{max} \}$$

4) two children nodes are created: the left node corresponds to points xᵢ<x*ᵢ the other with <CWU-Call number

$${x}_{i} \geq {x}_{i}^{*} .$$

5) the procedure from 2 to 4 is repeated until a data point is isolated (the node has zero children node).
   Then, the anomaly score for every point x∈X is a function of the length of the path from the root to x.

The parameters to be tuned when using iForest are ψ, the number of data of the sub-sample, and T, the number of trees in the forest. In this example the parameters were set to their default values (ψ=100, T=256).

Yet another illustrative unsupervised anomaly detection algorithm that was employed is the Histogram-Based Outlier Score (HBOS), which is one of the simplest and well known statistical anomaly detection algorithms and which uses histograms to compute the anomaly score.

The algorithm works as follows: consider a dataset X⊂R^{F}, where Fis the number of features. For each feature i∈[1, . . . , F], the univariate histogram is created and normalized.

Then, to evaluate the anomaly score for every point x∈X, each of its feature values xᵢ∈[1, . . . F] is considered. The height of the bin histᵢ(xᵢ) where xᵢ falls is extracted. Then the anomaly score AS_{HOBS}(x) is defined as

$${AS}_{HOBS} ( x ) = {\sum }_{i = 0}^{F} log ( \frac{1}{{hist}_{i} ( {x}_{i} )} ) \qquad ( 4 )$$

Both fixed-width or dynamic-width bin histograms can be used and in this example a dynamic-width bin histogram was employed. To determine the size of the bin, values are sorted and fixed N/k successive values are grouped into a single large bin, where N is the number of data-points in the dataset and k the number of bins. In the histogram that is constructed, since the area of each bin is equal to the number of items falling in the bin, it is fixed to N/k. Therefore, the height of the bin is the reciprocal of the width of the bin. If a bin has a small height, the bin covers a large interval of the value and thus represents data with low density. Therefore, it is more likely to be anomalous.

### Alert Generation

As the final step of the anomaly detection process, an anomaly score threshold needs to be selected to generate an alert such as an alarm. This can be accomplished, in one embodiment, by splitting the dataset into a training set and test set (80:20) and defining a grid of possible thresholds and a cost function as follows:

$$J ( thr ) = {[ 1 - Recall ( thr ) ]}^{2} + {[ FP / day ( thr ) ]}^{2} . \qquad ( 5 )$$

where J represents the Euclidean distance of a point on the FP/dayRecall curve from the bottom right corner, which is the point where the ideal performance was achieved (Recall=1; FP/day=0). For each threshold, the average recall and FP/day is computed for the training set and the corresponding value of J.

Once all the possible thresholds of the grid are considered, the one that minimizes the cost function J is selected:

$${threshold}_{opt} = arg min ( J ) \qquad ( 6 )$$

One significant advantage that arises from the use of an unsupervised algorithm is that labeling is not required. However, this can limit the discriminating power of these approaches so that they identify all anomalous phenomena even if they are not the type of fault that are of interest. That is, an unsupervised approach can lead to the detection of false positives.

In some embodiments this problem can be mitigated by use of a Root Cause Analysis (RCA) procedure, which leverages a-priori information to reduce the number of false positive instances related to other sources of anomalies.

In particular, the RCA procedure showed that several false positives are generated in connection with possible hypoglycemic treatments or meals and with particularly noisy regions of the CGM trace. Thus, these false positives can be suppressed during:
A prolonged and rapid decrease in the glucose concentration (e.g., d′cgm<1 [mg/dL/min] for more than 2 hours); or
Noisy portions of the CGM sections where the difference between d′cgm(t) and the average of d″cgm(t) computed over the whole CGM trace is higher than e.g., 0.2 mg/dL/s².

In general, the thresholds used when either of these conditions are satisfied can be selected by visually inspecting the recurrent characteristics of FPs, TPs and FNs and selecting some values that provide reasonable tradeoffs among them.

### Illustrative Validation of the Systems and Methods Described Herein

The following section illustrates that the systems and methods described herein are effective for retrospective in detecting PISA. To assess the performance of the PISA detection method described herein, the output of the algorithm is compared against a ground truth label specifying the presence or absence of a PISA artifact. Unfortunately, highly reliable labels of this kind are not commonly available on real-data. Therefore, two datasets are considered: a simulated dataset and a retrospectively labelled real dataset.

The simulated dataset is obtained by generating CGM traces with an accurate simulator of TID patient physiology, in particular the UVa/Padova TID Simulator. PISAs are then included using a realistic model of this fault. Since the faults are applied in known positions and for a known duration, the exact label is available for the simulated dataset.

Even using an accurate simulator, however, synthetic data offer an unavoidably simplified representation of the complex physiological fluctuation occurring in real glycemic traces. For this reason, a real dataset of 72 CGM traces was also used in the assessment of the PISA detection method. In this case, data was retrospectively labelled by expert human operators. Unfortunately, labeling is not a trivial task even for human experts and this introduces a certain degree of uncertainty in the ground truth.

### Simulated Data

First, the method was tested on a simulated dataset of N=100 patients wearing the CGM sensor for 10 days. This data were generated using the UVA/Padova type 1 diabetes simulator, an accurate computer simulator of the TID physiology, accepted by the Food and Drugs Administration (FDA) as an alternative of the pre-clinical animal trails.

For each of the 100 simulated subjects, a protocol with 3 meals per day was simulated. Meal times are randomly chosen in the time intervals of [7.00-8.00], [11.30-13.00] and [18.30-20.00] while the relative amounts are sampled according to a previously determined distribution. At each meal, the patient is treated with an insulin dose proportional to the estimated amount of carbohydrates contained in the meal. Apart from meal-time, insulin delivery was adjusted by a hybrid closed-loop algorithm based on a proportional integral derivative (PID) control strategy. Each CGM trace generated with the simulator is corrupted with realistic measurements noise.

Compression artifacts are simulated using a previously proposed model: each fault is modelled as an additive error on the CGM signal, obtained by filtering a rectangular function with duration D and unitary amplitude, that models the presence of mechanical pressure on the sensor through a first order linear system that accounts for the delayed impact of the pressure on the CGM sensor. The first-order linear system has a transfer function G(s)=−P/(1+τs), where P is linked to the maximum amplitude (in mg dL) of the episode Aₘₐₓ,

$${A}_{max} = P ( 1 - {e}^{- \frac{D}{τ}} )$$

and τ the time constant of the system, can be thought of as one-third of the time it takes the artifact to dissipate after the pressure is released. The values of P, τ and D are extracted from Gaussian distributions.

The number of faulty episodes for each patient is extracted from a truncated Gaussian distribution. Specifically, it is obtained by sampling from a Gaussian distribution with mean 3, standard deviation of 3 and resampling values lower than 1 or greater than 10. Timing of the episodes is chosen from a randomly uniform distribution (with resampling in case of overlapping episodes)

The dataset is split into a training set and test set consisting of 80 and 20 subjects respectively.

### Real Data

Real data typically lacks accurate information about the presence of PISA faults. In fact, it is difficult to induce a controlled PISA event, even in a dedicated experiment. An alternative option is the manual labeling by clinicians and domain experts through a-posteriori visual inspection of a CGM trace. Nevertheless, the resulting label is operator dependent and disagreements among operators are not infrequent. Moreover, the process is time consuming, limiting the availability of manually labelled datasets.

To assess the PISA detection method described herein a real dataset consisting of 72 CGM traces (extracted from a larger multi-center study) was collected from 36 adult subjects with Type 1 Diabetes whose glucose concentrations were monitored for 10 days using the Dexcom G6 CGM sensor (Dexcom Inc., San Diego, CA). This dataset has been retrospectively manually labelled. Only subjects simultaneously wearing two different CGM devices were selected so that the comparison between the two traces can be used to facilitate the detection of PISA episodes. It should be noted that the presence of the two simultaneous CGM traces is only used for the definition of the ground-truth, while the detection method used only one of the two signals.

The ground-truth is defined knowing that the failures are characterized by a rapid fall in glucose level (due to the pressure applied on the sensor) and a fast consequent recovery (when the pressure is released) that affect only one device of the two worn by the subject. However, the visual recognition of the malfunctioning is a not trivial task because the signal can assume a shape that is similar to the artifacts under certain physiological and normal conditions.

The traces were visually inspected by 3 operators and a labelled technique using a 3-level scale of confidence in the recognition of the fault was applied. In this technique, the higher the label assigned, the higher the certainty that the portion of the signal is a compression artifact (further details are provided in Table II). Consensus meetings with 3 senior operators were arranged to solve discrepancies in the manual labeling process. Finally, only the portions of the signal with a label greater than 2 were selected as faulty.

TABLE II

| Label | Meaning |
|---|---|
| 1 | Probably not a PISA |
| 2 | It could a PISA |
| 3 | It is a PISA |

### Evaluation Criteria

The effectiveness of the PISA detection methods described herein is evaluated basing on the count of the true positives (TPs), the false negatives (FNs) and the false positives (FPs).

If an alert is generated during a compression artifact, a true positive (TP) is assigned, otherwise the faulty portion of the CGM trace is classified as a false negative (FN). For all the other portions of the CGM trace, if an alert is incorrectly generated, a false positive (FP) is assigned, otherwise a true negative (TN) is assigned. Moreover, since faults cause the system to remain in an anomalous state for some period of time, if an alert is generated within a 25-minutes time window after a fault, it is not counted as a false positive.

Then, the recall (also known as sensitivity) in the population is computed as:

$$Recall = \frac{TP}{TP + FN} \qquad ( 7 )$$

This metric represents the fraction of compression artifacts correctly detected. Moreover, the recall is also computed for each subject and its distribution over the population determined. Similarly, the number of false positives per day (FP/day) in the population is determined. Moreover, the FP/day is also computed for each subject and its distribution over the population determined.

True negative events and the associated metrics (e.g. specificity and false positive rates) are of limited interest in such an unbalanced dataset where the events to be detected occur infrequently with respect to not-faulty data. Finally, for the two evaluation metrics that are considered, the average value in the population is determined.

### Simulated Data Results

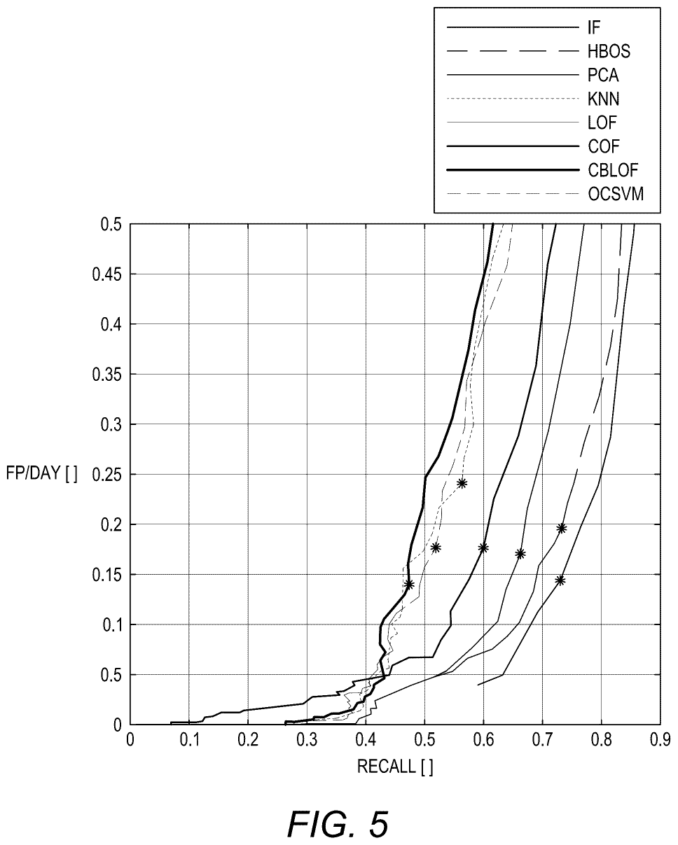

FIG. 5 shows FP/day-recall curves using the training dataset. Each point on the curve is obtained by considering a different anomaly score threshold and different curves correspond to different algorithms. The performance with the optimal threshold, computed on the training set as described below, is indicated by the black dot.

Once the threshold is computed it can be evaluated using the test dataset. The results are summarized in Table I.

The anomaly detection algorithms that were tested exhibit recalls between 60% and 70%, with a confidence frequently overlapping the confidence intervals. As such, no methods emerge as clearly superior to the others in detecting PISAs. The highest recall is observed with the Isolation Forest algorithm (74%), which achieves this result while generating only 0.17 FP/day, which is similar to or slightly better than the other anomaly detection algorithms. The lowest occurrence of FPs is observed with CBLOF (0.14), which on average achieved about 1.4 FP during the 10 days of monitoring: however, the method also shows inferior discriminatory power in terms of the number of faults that are recognized (recall equal to 51%).

Since the Isolation Forest algorithm appears to exhibit the most appealing trade-off between recall and false positives per day, additional analysis provided below will focus on the results obtained with this algorithm.

TABLE III
COMPARISON OF THE PARAMETERS OF THE FAULTS

| | P | D | τ | Amax |
|---|---|---|---|---|
| | [mg/dL] | [min] | [min] | [mg/dL |
| True Positives | 46 | 20 | 6 | 43.7 |
| | [37, 60] | [15, 25] | [5, 8] | [35.2, 57.0] |
| False Negatives | 30 | 25 | 8 | 28.5 |
| | [20, 40] | [20, 35] | [7, 12] | [19.0, 38.0] |

Results expressed as median [25th-75th percentiles]

Table III shows a comparison between the parameters (P,D,τ,Aₘₐₓ) of the detected faults (i.e. TP) and the parameters of the faults that are undetected. As expected, the algorithm tends to more easily detect PISAs with a larger maximum amplitude Aₘₐₓ and those resulting from the application of greater pressure. Moreover, the algorithm tends to more easily detect PISAs that produce more abrupt pressure changes.

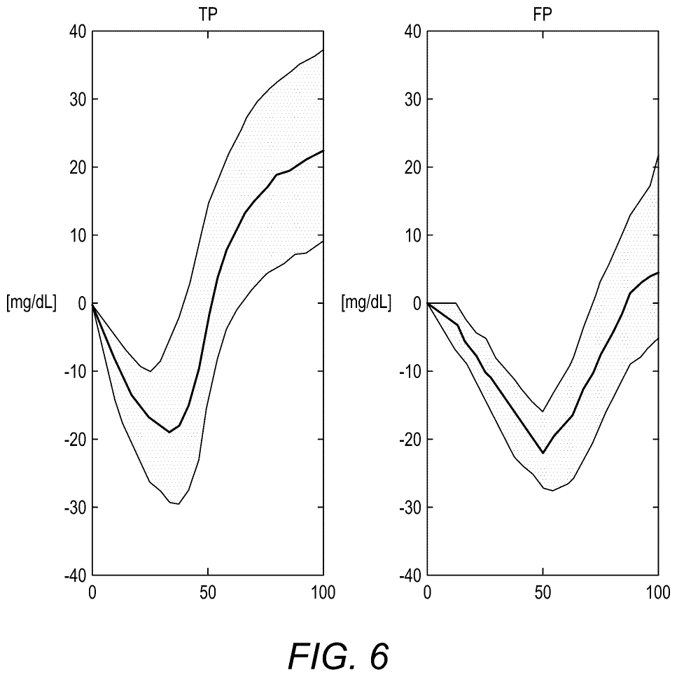

To compare the shape of CGM traces during TPs and FPs, FIG. 6 shows a normalized representation of portions of the CGM trace detected with the Isolation Forest algorithm using the training dataset of the simulated data. More precisely, to make them comparable, the CGM traces are first aligned along y=0 by subtracting the first CGM value and then plotting the results as a function of the percentage of the duration period that has transpired, where 0% is the starting point and 100% is the final sample. FIG. 6 also shows the median (black line) and the 25 and 75 percentiles (dashed line). The shape of the CGM traces are very similar in both cases, i.e., the PISA can be easily confused with a physiological fluctuation of the glucose concentration. This illustrates the challenges involved in detecting PISAs.

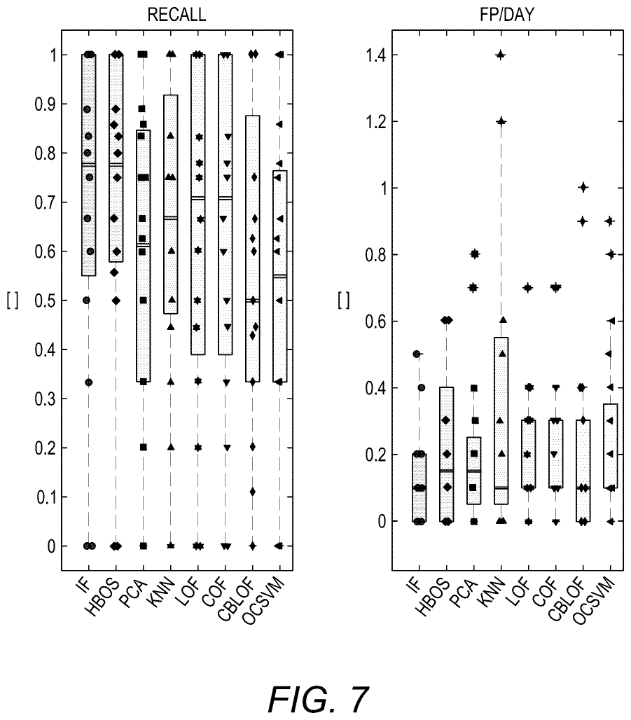

FIG. 7 shows a boxplot of the population distributions of the recall and FP/day metrics in the test dataset. A scatter plot is also superimposed, where each dot represents the performance of one test subject. The figure shows that the Isolation Forest algorithm scores a recall higher than 50% and a FP/day lower than 0.2 (i.e., 2 FPs over 10 days of monitoring) in all the subjects except for three.

### Real Data

In real patients, compression artifacts are more likely to occur during the night when the patient is sleeping and can unintentionally exert a prolonged pressure on the sensor. The distribution of the occurrence of PISAs according to time of day, even if observed, has never been modelled in the literature. Thus, this variability is not introduced in the generation of faults in the simulated scenario but is considered for the real data. In particular, the procedure for the selection of the optimal threshold is repeated twice to tune two different thresholds for different time slots: one is used for the diurnal time (from 7 am to 11 pm) and the other is used during the night (from 11 pm to 7 am). In this way, each algorithm is characterized by two different degrees of aggressiveness: in fact, the algorithm can be less aggressive during the night, when faults are easier to detect with respect to the faults occurring during the day. Indeed, during the day, many fluctuations and other sources of anomalies (e.g., meal, snacks, physical activity, etc.) can make their detection more complex. The use of two thresholds was adopted to test the algorithms proposed herein for PISA detection in the simulated scenario. The performance results that were achieved are summarized in in Table V.

TABLE IV

| | Recall | FP/day |
|---|---|---|
| Algorithm | [ ] | [ ] |
| Principal Component | 0.57 | 0.35 |
| Analysis | | |
| Isolation Forest | 0.55 | 0.29 |
| Histogram-Based Outlier | 0.54 | 0.41 |
| Score | | |
| One-Class Support Vector | 0.49 | 0.36 |
| Machine | | |
| K-Nearest Neighbors | 0.45 | 0.29 |
| Clustering-Based Local | 0.42 | 0.26 |
| Outlier Factor | | |
| Local Outlier Factor | 0.39 | 0.29 |
| Connectivity Outlier Factor | 0.31 | 0.47 |

Confirming the in silico findings, some of the highest recalls are obtained by the IForest and HBOS algorithms (0.55 and 0.54, respectively) but HBOS generate more than 4 FPs in 10 days while IForest provides one of the lowest numbers of false alarms (less than 3 in 10 days). Higher recall is achieved only by PCA, which recognizes 57% of the faults while producing 3-4 FPs during the 10 days of monitoring. As in the simulated scenario, the CBLOF algorithm exhibits the lowest FP/day but also exhibits a limited discriminating power (recall equal to 0.42).

In other words, despite the similarity between PISAs and physiological glucose fluctuations, in the population under study the detection methods described herein allow more than 50% of these anomalies to be detected and discarded, preventing possible erroneous decisions caused by incorrect data. This result is achieved while limiting the fraction of correct CGM data that is erroneously discarded: on average, 3 to 4 FPs occur over 10 days of monitoring, corresponding to about 130-180 minutes of monitoring that are unnecessarily discarded (0.9%-1.25% of the 14400 minutes of monitoring available).

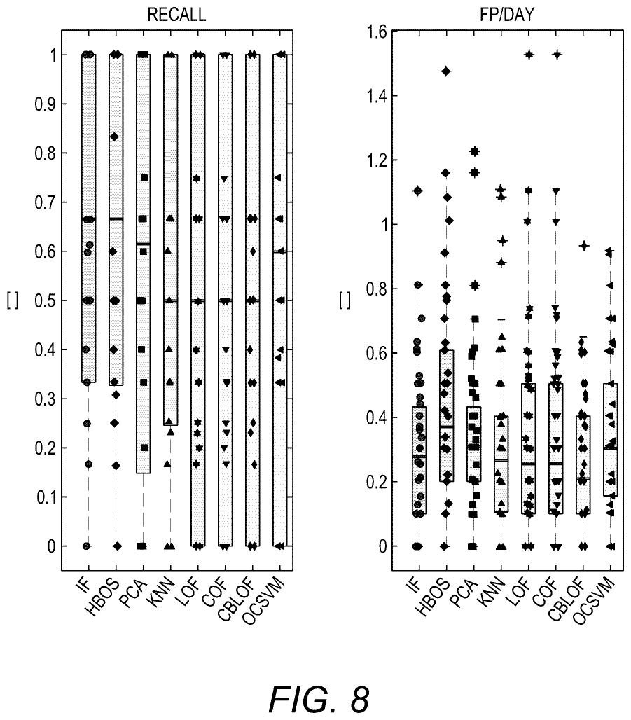

FIG. 8 shows boxplots of the distribution of the recall and FP/day metrics in patients. Regarding the simulated data, a scatter plot is also superimposed on the boxplot, where each dot represents the performance in one test subject). It can be observed that, even if the recall achieved in the population for the Forest algorithm is 55% (as shown in Table V), 75% of the subjects achieve a recall higher than 0.5 with a median of 0.76. This result suggests that the algorithm struggles with a fraction of particularly challenging traces (recall 0.3 in 12.5% of the traces), possibly affected by high noise or large glycemic fluctuation. Similar conclusions can be made for all the algorithms tested.

TABLE V

| | Recall | FP/day |
|---|---|---|
| Settings | [ ] | [ ] |
| Aggressive | 0.90 | 3.60 |
| | [0.83- | [3.19- |
| | 0.94] | 4.07] |
| Nominal | 0.80 | 2.11 |
| | [0.72- | [1.82- |
| | 0.88] | 2.49] |
| Cautious | 0.67 | 1.30 |
| | [0.58- | [1.09- |
| | 0.76] | 1.63] |

Results expressed as mean [90% CI]

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

The claimed subject matter may be implemented as a method, apparatus, or article of manufacture using standard programming and/or engineering techniques to produce software, firmware, hardware, or any combination thereof to control a computer to implement the disclosed subject matter. For instance, the claimed subject matter may be implemented as a computer-readable storage medium embedded with a computer executable program, which encompasses a computer program accessible from any computer-readable storage device or storage media. For example, computer readable storage media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). However, computer readable storage media do not include transitory forms of storage such as propagating signals, for example. Of course, those skilled in the art will recognize many modifications may be made to this configuration without departing from the scope or spirit of the claimed subject matter.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. An unsupervised method of detecting a pressure induced sensor artifact (PISA) in an analyte signal, comprising:
receiving, by a processor, an analyte trace having a plurality of data samples obtained over a period of time;
extracting, by the processor, from the analyte trace a plurality of predetermined features reflective of a PISA in the analyte trace to produce a plurality of data points in a feature space defined by the plurality of predetermined features;
analyzing, by the processor, the data points in the feature space using an unsupervised anomaly detection algorithm to produce an anomaly score for each of the data points;
identifying, by the processor, data points that have an anomaly score that exceeds a threshold; and
generating, by the processor, an alert indicating that a PISA is present in data samples associated with the data points that have an anomaly score that exceeds the threshold.

2. The method of claim 1 wherein the plurality of predetermined features reflective of a PISA in the analyte trace are selected from the group consisting of a deviation from the local values (DLV) feature, a large non-monotonic variation (LNMV) feature, a deviation from the local trends (DLT) feature, and a first-down-then-up feature and a concavity feature.

3. The method of claim 1 wherein the unsupervised detection algorithm belongs to a class of algorithms selected from the group consisting of a nearest-neighbor based technique, a clustering-based method, a statistical algorithm and a random partitioning-based method.

4. The method of claim 1 wherein the unsupervised detection algorithm is an Isolation Forest algorithm or a Histogram-Based Outlier Score algorithm.

5. The method of claim 1 further comprising selecting the threshold by minimizing a cost function based on a recall metric and a number of false-positives per day metric.

6. The method of claim 1 further comprising preventing false positive alerts using a root cause analysis procedure based on a-priori information concerning noisy regions of the analyte trace or regions of the analyte trace in which analyte concentration is decreasing at a rate exceeding a threshold rate for a duration of time exceeding a threshold duration.

7. The method of claim 1 wherein producing the plurality of data points in the feature space defined by the plurality of predetermined features includes normalizing the extracted predetermined features such that each of the data points in the feature space are represented on a normalized scale.

8. A system for detecting a pressure induced sensor artifact (PISA) in an analyte signal, the system comprising:
an analyte sensor system configured to generate raw analyte data for a user;
a memory comprising executable instructions;
a processer in data communication with the memory and configured to execute the instructions to:
receive an analyte trace having a plurality of data samples obtained over a period of time;
extract from the analyte trace a plurality of predetermined features reflective of a PISA in the analyte trace to produce a plurality of data points in a feature space defined by the plurality of predetermined features;
analyze the data points in the feature space using an unsupervised anomaly detection algorithm to produce an anomaly score for each of the data points;
identify data points that have an anomaly score that exceeds a threshold; and
generate an alert indicating that a PISA is present in data samples associated with the data points that have an anomaly score that exceeds the threshold.

9. A computer-readable medium comprising instructions which, when executed by a processor, cause the processor to perform a method for detecting a pressure induced sensor artifact (PISA) in an analyte signal, noise reduction in analyte data, the method comprising:
receiving an analyte trace having a plurality of data samples obtained over a period of time;
extracting from the analyte trace a plurality of predetermined features reflective of a PISA in the analyte trace to produce a plurality of data points in a feature space defined by the plurality of predetermined features;
analyzing the data points in the feature space using an unsupervised anomaly detection algorithm to produce an anomaly score for each of the data points;
identifying data points that have an anomaly score that exceeds a threshold; and
generating an alert indicating that a PISA is present in data samples associated with the data points that have an anomaly score that exceeds the threshold.
